(19) United States
(12) Patent Application Publication
(10) Pub. No.: US 2009/0270474 A1
(43) Pub. Date: Oct. 29, 2009

(54) Therapeutic Agent for Keratoconjunctival Disorder

(76) Inventors: Keiichi Shibagaki, Nara (JP); Shin-ichiro Hirai, Nara (JP); Masatsugu Nakamura, Nara (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 12/085,214

(22) PCT Filed: Dec. 6, 2006
(86) PCT No.: PCT/JP2006/324324

§ 371 Date: May 19, 2008

(30) Foreign Application Priority Data
Dec. 6, 2005 (JP) 2005-341712

(51) Int. Cl. A61K 31/4164; A61P 27/02

(52) U.S. Cl. 514/396

(57) ABSTRACT

An object of the present invention is to discover a new use of eprosartan or a salt thereof. Eprosartan or a salt thereof exhibits an excellent improving effect in a corneal disorder model, and therefore is useful as a therapeutic agent for a keratoconjunctival disorder such as dry eyes, corneal ulcer, keratitis, conjunctivitis, superficial punctate keratopathy, corneal epithelial defects, conjunctival epithelial defects, keratoconjunctivitis sicca, superior limbic keratoconjunctivitis and filamentary keratitis.

### TECHNICAL FIELD

The present invention relates to a therapeutic agent for a keratoconjunctival disorder such as dry eyes, corneal ulcer, keratitis, conjunctivitis, superficial punctate keratopathy, corneal epithelial defects, conjunctival epithelial defects, keratoconjunctivitis sicca, superior limbic keratoconjunctivitis or filamentary keratitis, comprising eprosartan ((E)-a-[[2-n-butyl-1-[(4-carboxyphenyl)methyl]-1H-imidazol-5-yl]methylene]-2-thiophenepropionic acid) or a salt thereof as an active ingredient.

### BACKGROUND ART

Cornea is a transparent avascular tissue having a diameter of about 1 cm and a thickness of about 1 mm, while conjunctiva is a mucosal membrane covering the eyeball surface posterior to the corneal margin, and the back face of the eyelid. The cornea and the conjunctiva are known to significantly affect the visual function. Keratoconjunctival disorders caused due to a variety of diseases such as corneal ulcer, keratitis, conjunctivitis, dry eyes and the like may adversely affect normal architecture of corneal epithelium, and furthermore, may impair structures and functions of the corneal stroma and corneal endothelium when the repair of these disorders is retarded, alternatively when these disorders are prolonged without making repair on some grounds. That is because the cornea and the conjunctiva are connected tissues. In these years, with the development of cell biology, factors involved in cell proliferation, migration, adhesion, extension, differentiation and the like had been elucidated, and it was reported that these factors play important roles in repair of keratoconjunctival disorders (Japanese Review of Clinical Opthalmology, 46, 738-743 (1992), and Ophthalmic Surgery, 5, 719-727 (1992)).

On the other hand, JP-B-7-68223 discloses that eprosartan inhibits the action of angiotensin II and is useful as a therapeutic agent for hypertension, heart failure and the like.

However, there has been no report in which a pharmacological effect of eprosartan on a keratoconjunctival disorder is studied.

### DISCLOSURE OF THE INVENTION

### Problems to be Solved

Accordingly, it is a very interesting subject to discover a new medicinal use of eprosartan or a salt thereof.

### Means for Solving the Problems

The present inventors have made intensive studies in order to discover a new medicinal use of eprosartan, and as a result, they found that eprosartan mesylate exerts an excellent improving effect on a corneal disorder in a test for a therapeutic effect using corneal disorder models and thus the present invention has been completed.

That is, the present invention is directed to a therapeutic agent for a keratoconjunctival disorder such as dry eyes, corneal ulcer, keratitis, conjunctivitis, superficial punctate keratopathy, corneal epithelial defects, conjunctival epithelial defects, keratoconjunctivitis sicca, superior limbic keratoconjunctivitis or filamentary keratitis, comprising eprosartan (hereinafter referred to as “the present compound”) or a salt thereof as an active ingredient.

The salt of the present compound is not particularly limited as long as it is a pharmaceutically acceptable salt, and examples thereof include sodium salts, potassium salts, lithium salts, calcium salts, magnesium salts, salts with an inorganic acid such as hydrochloric acid, nitric acid or sulfuric acid, salts with an organic acid such as acetic acid, fumaric acid, maleic acid, succinic acid, tartaric acid, methanesulfonic acid or paratoluenesulfonic acid, and the like. Quaternary ammonium salts are also included in the salt according to the present invention. More preferred salts are methanesulfonic acid salts, sodium salts and potassium salts. When the present compound and the salt thereof are present in the form of crystals, their crystalline polymorphisms are also included in the scope of the present invention. When there are geometric isomers of the present compound, these isomers are also included in the scope of the present invention. Further, the present compound may be in the form of a hydrate or a solvate.

The keratoconjunctival disorder as used herein means the state of damaged cornea and/or conjunctiva due to various causes, and examples thereof include dry eyes, corneal ulcer, keratitis, conjunctivitis, superficial punctate keratopathy, corneal epithelial defects, conjunctival epithelial defects, keratoconjunctivitis sicca, superior limbic keratoconjunctivitis, filamentary keratitis and the like.

The therapeutic agent for a keratoconjunctival disorder of the present invention may be administered either orally or parenterally.

Examples of the dosage form include eye drops, ophthalmic ointments, injections, tablets, capsules, granules, powders and the like. In particular, eye drops are preferred. These can be prepared using any of generally used techniques. For example, the eye drops can be prepared using a tonicity agent such as sodium chloride or concentrated glycerin, a buffer such as sodium phosphate or sodium acetate, a surfactant such as polyoxyethylene sorbitan monooleate, polyoxyl 40 stearate or polyoxyethylene hydrogenated castor oil, a stabilizer such as sodium citrate or sodium edetate, a preservative such as benzalkonium chloride or paraben as needed. The pH of the eye drops is permitted as long as it falls within the range that is acceptable as an ophthalmic preparation, but is preferably in the range of from 4 to 8.

The ophthalmic ointments can be prepared with a generally used base such as white soft paraffin or liquid paraffin. Also, oral preparations such as tablets, capsules, granules and powders can be prepared by adding an extender such as lactose, crystalline cellulose, starch or vegetable oil, a lubricant such as magnesium stearate or talc, a binder such as hydroxypropyl cellulose or polyvinyl pyrrolidone, a disintegrant such as carboxymethyl cellulose calcium or low-substituted hydroxypropylmethyl cellulose, a coating agent such as hydroxypropylmethyl cellulose, macrogol or a silicone resin, a film forming agent such as gelatin film, and the like, as needed.

The present invention also provides a method for treating a keratoconjunctival disorder comprising administering to a patient a therapeutically effective amount of eprosartan or a salt thereof.

The dose of the present compound can properly be selected depending on the symptoms, age, dosage form and the like. In the case of an eye drop, it may be instilled once to several times a day at a concentration of from 0.00001 to 10% (w/v), preferably from 0.001 to 3% (w/v). In the case of an oral preparation, it may be administered once or divided into several times at a dose of generally from 0.1 to 5000 mg per day, preferably from 1 to 1000 mg per day.

### ADVANTAGE OF THE INVENTION

As will be described below, when a test for a therapeutic effect on a corneal disorder was carried out, eprosartan mesylate ((E)-a-[[2-n-butyl-1-[(4-carboxyphenyl)methyl]-1H-imidazol-5-yl]methylene]-2-thiophenepropionic acid monomethanesulfonate) was found to exert an excellent improving effect on corneal disorder models. Therefore, eprosartan or a salt thereof is useful as a therapeutic agent for a keratoconjunctival disorder such as dry eyes, corneal ulcer, keratitis, conjunctivitis, superficial punctate keratopathy, corneal epithelial defects, conjunctival epithelial defects, keratoconjunctivitis sicca, superior limbic keratoconjunctivitis, filamentary keratitis and the like.

### BEST MODE FOR CARRYING OUT THE INVENTION

Hereinafter, results of a pharmacological test and preparation examples will be shown, however, these examples are for understanding the present invention well, and are not meant to limit the scope of the present invention.

### [Pharmacological Test]

### Test For Therapeutic Effect On Corneal Disorder

Using male SD rats, corneal disorder models were produced in accordance with the method of Fujihara et al. (Invest. Opthalmol. Vis. Sci. 42 (1): 96-100 (2001)). After the production of the corneal disorder models, the corneal disorder score was evaluated in accordance with the method of Murakami et al. (Journal of the eye 21 (1): 87-90 (2004)), and the improvement ratio of corneal disorder after instillation of eprosartan mesylate (hereinafter referred to as “Compound A”) was obtained.

### (Test Method)

As test animals, male SD rats were used, and systemic anesthesia was given to the rats by administration of Nembutal. Subsequently the exorbital lacrimal gland was removed and a corneal disorder was induced over a period of 2 months.

Then, Compound A was administered as follows.

### Compound A Instillation Group:

A phosphate-buffered saline solution (PBS solution) containing Compound A (0.1%) was instilled into both eyes 6 times a day for 14 days (instilled amount: 5 μL/dose) (one group consisting of 4 animals, 8 eyes).

In a control group, PBS solution was instilled into both eyes 6 times a day for 14 days (instilled amount: 5 μL/dose) (one group consisting of 4 animals, 8 eyes).

Fourteen days after the start of instillation, the damaged parts of the cornea were stained with fluorescein. For each of the upper, middle and lower parts of the cornea, the degree of fluorescein staining was evaluated by scoring according to the criteria shown below and the improvement ratio of corneal disorder was calculated from the mean value of the total scores for each of the above-mentioned parts.

Also for normal eyes, the mean value of the total scores for each of the above-mentioned parts was obtained.

### (Evaluation Criteria)

0: No punctate staining
1: Scattered staining (punctate, separated staining)
2: Moderate staining (a part of punctate staining being adjacent)
3: Heavy staining (punctate, barely separated staining)

### (Results)

By taking the mean value of the total scores for the control group (PBS solution) as a standard (improvement ratio: 0%) and according to the calculation formula shown below, the improvement ratio in the Compound A instillation group was calculated, which is shown in Table 1. Incidentally, the mean value of the scores is a mean of those of 8 cases, respectively.

Improvement ratio (%)={(control)−(Compound A)}/damage degree×100

Damage degree=(control)−(normal eye)

TABLE 1

| | Mean value | Improvement |
|---|---|---|
| | of scores | ratio (%) |
| Normal eye | 2.8 | |
| Control group | 6.1 | |
| Compound A instillation group (0.1%) | 3.3 | 84.8 |

### (Discussion)

As apparent from the results of the above pharmacological test using rats (Table 1), Compound A significantly improved a corneal damage.

### Preparation Examples

Hereinafter, representative preparation examples using Compound A will be shown.

### Formulation Example 1

In 100 ml,

| | |
|---|---|
| Compound A | 10 mg |
| Sodium Chloride | 900 mg |
| Sterile purified water | q.s. |

By altering the amount of Compound A to be added, an eye drop at a concentration of 0.001% (w/v), 0.03% (w/v), 0.1% (w/v), 0.3% (w/v), 1.0% (w/v), or 3.0% (w/v) can be prepared.

### Formulation Example 2

In 100 g,

| | |
|---|---|
| Compound A | 0.3 g |
| Liquid paraffin | 10.0 g |
| White soft paraffin | q.s. |

By altering the amount of Compound A to be added, an ophthalmic ointment at a concentration of 1% (w/w) or 3% (w/w) can be prepared.

### INDUSTRIAL APPLICABILITY

Eprosartan or a salt thereof is useful as a therapeutic agent for a keratoconjunctival disorder such as dry eyes, corneal ulcer, keratitis, conjunctivitis, superficial punctate keratopathy, corneal epithelial defects, conjunctival epithelial defects, keratoconjunctivitis sicca, superior limbic keratoconjunctivitis and filamentary keratitis.

1. A therapeutic agent for a keratoconjunctival disorder, comprising eprosartan or a salt thereof as an active ingredient.

2. The therapeutic agent for a keratoconjunctival disorder according to claim 1, wherein the keratoconjunctival disorder is dry eyes, corneal ulcer, keratitis, conjunctivitis, superficial punctate keratopathy, corneal epithelial defects, conjunctival epithelial defects, keratoconjunctivitis sicca, superior limbic keratoconjunctivitis or filamentary keratitis.

3. The therapeutic agent for a keratoconjunctival disorder according to claim 1, wherein the dosage form of the agent is an eye drop or an ophthalmic ointment.

4. A method for treating a keratoconjunctival disorder comprising administering to a patient a therapeutically effective amount of eprosartan or a salt thereof.

5. The method for treating a keratoconjunctival disorder according to claim 4, wherein the keratoconjunctival disorder is dry eyes, corneal ulcer, keratitis, conjunctivitis, superficial punctate keratopathy, corneal epithelial defects, conjunctival epithelial defects, keratoconjunctivitis sicca, superior limbic keratoconjunctivitis or filamentary keratitis.

6. The method for treating a keratoconjunctival disorder according to claim 4, wherein the administration form of eprosartan or a salt thereof is an eye drop or an ophthalmic ointment.

7. Use of eprosartan or a salt thereof for manufacturing a therapeutic agent for a keratoconjunctival disorder.

8. The use according to claim 7, wherein the keratoconjunctival disorder is dry eyes, corneal ulcer, keratitis, conjunctivitis, superficial punctate keratopathy, corneal epithelial defects, conjunctival epithelial defects, keratoconjunctivitis sicca, superior limbic keratoconjunctivitis or filamentary keratitis.

9. The use according to claim 7, wherein the dosage form of the agent is an eye drop or an ophthalmic ointment.

10. The therapeutic agent for a keratoconjunctival disorder according to claim 2, wherein the dosage form of the agent is an eye drop or an ophthalmic ointment.

11. The method for treating a keratoconjunctival disorder according to claim 5, wherein the administration form of eprosartan or a salt thereof is an eye drop or an ophthalmic ointment.

12. The use according to claim 8, wherein the dosage form of the agent is an eye drop or an ophthalmic ointment.
